(19) United States
(12) Patent Application Publication
(10) Pub. No.: US 2010/0111318 A1
(43) Pub. Date: May 6, 2010

(54) ACTIVE NOISE CONTROLLER

(75) Inventor: Yoshio Nakamura, Osaka (JP)

Correspondence Address: PEARNE & GORDON LLP, 1801 EAST 9TH STREET, SUITE 1200, CLEVELAND, OH, 44114-3108, US

(73) Assignee: PANASONIC CORPORATION, Osaka (JP)

(21) Appl. No.: 12/532,498

(22) PCT Filed: Mar. 25, 2008
(86) PCT No.: PCT/JP2008/000704

§ 371 Date: Sep. 22, 2009

(30) Foreign Application Priority Data
Mar. 30, 2007 (JP) 2007-093241

(51) Int. Cl. A61F 11/06

(52) U.S. Cl. 381/71.8

(57) ABSTRACT

An active noise controller using an adaptive notch filter performs a coefficient update operation by using only a signal obtained by processing an error signal with ½ cycle of the frequency of noise to be reduced for a predetermined period. This eliminates the necessity of product operation for the coefficient update operation, and in turn significantly reduces an operation load.

### TECHNICAL FIELD

The present invention relates to an active noise reduction device for actively reducing vibratory noise generated by a rotating machine such as an engine of a vehicle.

### BACKGROUND ART

For a conventional active noise reduction device, patent document 1 discloses an adaptive control method using an adaptive notch filter.

FIG. 7 is a block diagram showing a configuration of a conventional active noise reduction device. In FIG. 7, discrete operation processing unit 115 executes a discrete operation for implementing the active noise reduction device. Engine rotational speed detector 101 outputs a pulse train as engine pulse p, which has a frequency proportional to an engine rotational speed. For example, engine pulse p is generated by extracting an output of a crank angle sensor. Frequency detecting unit 102 calculates and outputs noise frequency f based on engine pulse p. Basic signal generating unit 116 has sine wave table 103 holding, in a memory, a value of each point defined by equally dividing one cycle of a sine wave into predetermined parts. Selection unit 117 selects data from sine wave table 103, and generates and outputs basic sine wave signal x1[n] and basic cosine wave signal x2[n] having the same frequency as noise frequency f. Reference signal generating unit 118 generates and outputs reference sine wave signal r1[n] and reference cosine wave signal r2[n] by using basic sine wave signal correction value table 119 (a basic sine wave signal correction value in a frequency of f (Hz) is referred to as C1[f]) and basic cosine wave signal correction value table 120 (a basic cosine wave signal correction value in a frequency of f (Hz) is referred to as C2[f]) simulating a transfer characteristic value from loudspeaker 110 to microphone 111.

First one-tap digital filter 107 filters basic sine wave signal x1[n] based on filter coefficient W1[n] held therein and generates first control signal y1[n]. Second one-tap digital filter 108 filters basic cosine wave signal x2[n] based on filter coefficient W2[n] held therein and generates second control signal y2[n]. Power amplifier 109 amplifies a signal obtained by adding first control signal y1[n] and second control signal y2[n] to each other. Loudspeaker 110 outputs an output signal from power amplifier 109 as a noise cancellation sound. Microphone 111 detects a sound generated as a result of interference of a noise and a noise cancellation sound as error signal ε[n].

First adaptive control algorithm operating unit 112 sequentially updates filter coefficient W1[n] by, for example, an LMS (Least Mean Square) algorithm that is a kind of steepest descent method based on reference sine wave signal r1[n] and error signal ε[n]. Similarly, second adaptive control algorithm operating unit 113 sequentially updates filter coefficient W2[n] based on reference cosine wave signal r2[n] and error signal ε[n].

Sequential updating formulae of coefficients W1 and W2 are represented by the following equations.

W1[n+1]=W1[n]−μ×r1[n]×ε[n] (1)

W2[n+1]=W2[n]−μ×r2[n]×ε[n] (2)

Herein, μ denotes a constant called a convergence coefficient and relates to a time period in which coefficients W1 and W2 converge on suitable values.

By repeating the above-mentioned processing with a predetermined cycle, noises can be reduced.

However, in generating reference sine wave signal r1[n] and reference cosine wave signal r2[n], the above-mentioned conventional configuration carries out a product-sum operation of basic sine wave signal x1[n] and basic sine wave signal correction value C1[k] as well as a product-sum operation of basic cosine wave signal x2[n] and basic cosine wave signal correction value C2[k]. It needs two product operations in order to generate the respective reference signals. Furthermore, in order to determine coefficients W1 and W2 of the one-tap digital filters, the above-obtained reference sine wave signal r1[n] and reference cosine wave signal r2[n] need to be multiplied by convergence coefficient μ and error signal ε[n], respectively. Therefore, two product operations are required (see, formulae (1) and (2)). That is to say, in order to determine coefficients W1 and W2 of the respective one-tap digital filters, four product operations are required. As a result, operation load increases.

[Patent document 1] Unexamined Japanese Patent Publication No. 2004-361721

### SUMMARY OF THE INVENTION

The present invention provides an active noise controller in which an operation load necessary for noise-suppression control is reduced by minimizing the execution of product operation.

An active noise controller in accordance with the present invention includes a frequency detecting unit for detecting a frequency of noise to be controlled from a noise source; a sine wave generating unit for generating a sine wave having the same frequency as the frequency of the noise detected by the frequency detecting unit; a cosine wave generating unit for generating a cosine wave having the same frequency as the frequency of the noise detected by the frequency detecting unit; a first one-tap digital filter to which a sine wave signal from the sine wave generating unit is input; a second one-tap digital filter to which a cosine wave signal from the cosine wave generating unit is input; an interference signal generating unit to which a noise control signal obtained by adding an output from the first one-tap digital filter and an output from the second one-tap digital filter to each other is input and which outputs an interference signal to cause interference with the noise to be controlled from the noise source; an error signal detecting unit for detecting an error signal generated as a result of the interference of the interference signal output from the interference signal generating unit and the noise to be controlled from the noise source; a first coefficient updating unit for updating a filter coefficient of the first one-tap digital filter; and a second coefficient updating unit for updating a filter coefficient of the second one-tap digital filter. The first coefficient updating unit and the second coefficient updating unit update the coefficients of the first one-tap digital filter and the second one-tap digital filter so that noise in the error signal detecting unit is reduced, based on a coefficient update signal obtained by processing the error signal from the error signal detecting unit with ½ cycle of the frequency of the noise to be controlled for a predetermined period.

### BRIEF DESCRIPTION OF THE DRAWINGS

FIG. 1 is a block diagram showing an active noise controller in accordance with a first exemplary embodiment of the present invention.

FIG. 2 is a graph showing an example of a sine wave table in the active noise controller in accordance with the first exemplary embodiment of the present invention.

FIG. 3 shows an example of a sine wave table in the active noise controller in accordance with the first exemplary embodiment of the present invention.

FIG. 4 is a graph showing an example of a transfer characteristic from a loudspeaker to a microphone of the active noise controller in accordance with the first exemplary embodiment of the present invention.

FIG. 5 shows an example of a characteristic table having a lower limit passing point and an upper limit passing point corresponding to a transfer characteristic from the speaker to the microphone shown in FIG. 4 in the active noise controller in accordance with the first exemplary embodiment of the present invention.

FIG. 6A is a graph showing a time-base waveform of a square wave formed by the active noise controller in accordance with the first exemplary embodiment of the present invention.

FIG. 6B is a graph showing a harmonic analysis of a square wave processed by the active noise controller in accordance with the first exemplary embodiment of the present invention.

FIG. 7 is a block diagram showing a configuration of a conventional active noise reduction device.

### REFERENCE MARKS IN THE DRAWINGS

1 engine rotational speed detector
2 frequency detecting unit
3 sine wave table
4 characteristic table
5 sine wave generating unit
6 cosine wave generating unit
7 first one-tap digital filter
8 second one-tap digital filter
9 power amplifier
10 loudspeaker (interference signal generating unit)
11 microphone (error signal detecting unit)
12 first adaptive control algorithm operating unit (first coefficient updating unit)
13 second adaptive control algorithm operating unit (second coefficient updating unit)
14 coefficient update signal generating unit
15 discrete operation processing unit

### DETAILED DESCRIPTION OF THE PREFERRED EMBODIMENT

### First Exemplary Embodiment

Hereinafter, an active noise controller in accordance with a first exemplary embodiment of the present invention is described with reference to drawings.

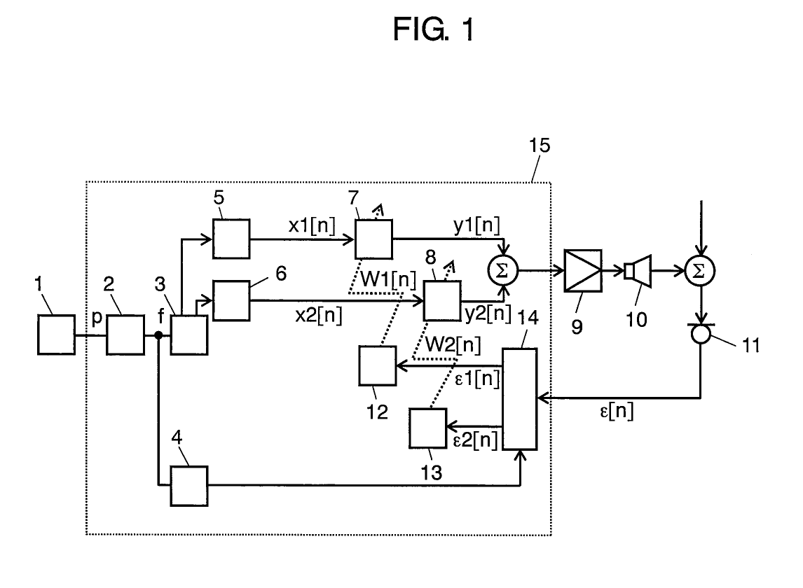

FIG. 1 is a block diagram showing an active noise controller in accordance with the first exemplary embodiment of the present invention. In FIG. 1, engine rotational speed detector 1 outputs a pulse train as engine pulse p, which has a frequency proportional to a rotational speed of a vehicle-mounted engine as a noise source. Frequency detecting unit 2 calculates and outputs frequency f [Hz] of noise to be controlled from engine pulse p. Sine wave table 3 including discrete sine wave data holds, in a memory, a sine value at each point defined by equally dividing one cycle of the sine wave into N parts. Sine wave generating unit 5 reads out data in a position at readout point P[n] from the sine wave table in every sampling cycle, and generates basic sine wave signal x1[n]. At this time, the difference (P[n+1]−P[n]) between P[n+1] that is a value subsequent to the readout point and P[n] that is a value of the current readout point is N×f×T when the sampling cycle is denoted by T and the frequency of noise to be controlled is denoted by f.

Similarly, cosine wave generating unit 6 reads out a point preceding the readout point of sine wave generating unit 5 by only N/4, that is, data in a position of P[n]+N/4 and generates basic cosine wave signal x2[n]. At this time, when each of readout points P[n] and P[n]+N/4 exceeds N, a point obtained by subtracting N from the readout point should be a new readout point.

Characteristic table 4 holds lower limit point PP1[f] and upper limit point PP2[f] of the readout point for processing error signal ε[n] in every frequency based on the phase characteristic as the transfer characteristic from loudspeaker 10 to microphone 11.

Coefficient update error signal generating unit 14 reads lower limit point PP1[f] and upper limit point PP2[f] in frequency f of noise to be controlled from characteristic table 4 based on frequency f of noise to be controlled; based on them, processes error signal ε[n] detected by microphone 11; and generates coefficient update signals ε1[n] and ε2[n].

Herein, ε1[n] satisfies the following equations:

ε1[n]=ε[n] when PP1[f]≦P[n]≦PP2[f] is satisfied, and

ε1[n]=−ε[n] when PP1[f]+N/2≦P[n]≦PP2[f]+N/2 is satisfied.

In the cases other than the above, the following equation is satisfied.

ε1[n]=0 (3)

Furthermore, ε[n] satisfies the following equations:

ε2[n]=ε[n] when PP1[f]+N/4≦P[n]≦PP2+N/4[f] is satisfied, and

ε2[n]=−ε[n] when NP1[f]+N×¾≦P[n]≦NP2+N×¾[f] is satisfied.

In the cases other than the above, the following equation is satisfied.

ε2[n]=0 (4)

Next, first one-tap digital filter 7 holds first filter coefficient W1[n] therein, and outputs first control signal y1[n] based on basic sine wave signal x1[n] and first filter coefficient W1[n]. Second one-tap digital filter 8 holds second filter coefficient W2[n] therein, and outputs second control signal y2[n] based on basic cosine wave signal x2[n] and second filter coefficient W2[n].

Power amplifier 9 amplifies a noise control signal obtained by adding first control signal y1[n] and second control signal y2[n] to each other. Loudspeaker 10 outputs an output signal from power amplifier 9 as a noise cancellation sound. Microphone 11 detects a sound, which is generated as a result of interference of noise caused by engine vibration to be controlled and the noise cancellation sound, as error signal ε[n].

First adaptive control algorithm operating unit 12 as a first coefficient updating unit sequentially updates filter coefficient W1[n] of first one-tap digital filter 7 by using coefficient update signal ε1[n]. Second adaptive control algorithm operating unit 13 as a second coefficient updating unit sequentially updates filter coefficient W2[n] of second one-tap digital filter 8 by using coefficient update signal ε[n]. Thus, discrete operation processing unit 15 includes software.

Next, a specific operation of the device is described.

The generation of basic sine wave signal x1[n], generation of basic cosine wave signal x2[n], generation of first control signal y1[n], generation of second control signal y2[n], detection of error signal ε[n], update of first filter coefficient W1[n], and update of second filter coefficient W2[n] are all executed in the same cycle. In the following description, this cycle is defined as T (second).

For example, frequency detecting unit 2 generates an interrupt in every rising edge of engine pulse p, measures a time period between the rising edges, and calculates frequency f of noise to be controlled based on the measurement results. Sine wave table 3 holds, in the memory, discrete data of the sine value at each point defined by equally dividing one cycle of the sine wave into N parts. When an arrangement that stores sine values from 0th point to (N−1)th point is represented by z[m] (0≦m<N), the following relational expression (5) is satisfied.

z[m]=sin(360°×m/N) (5)

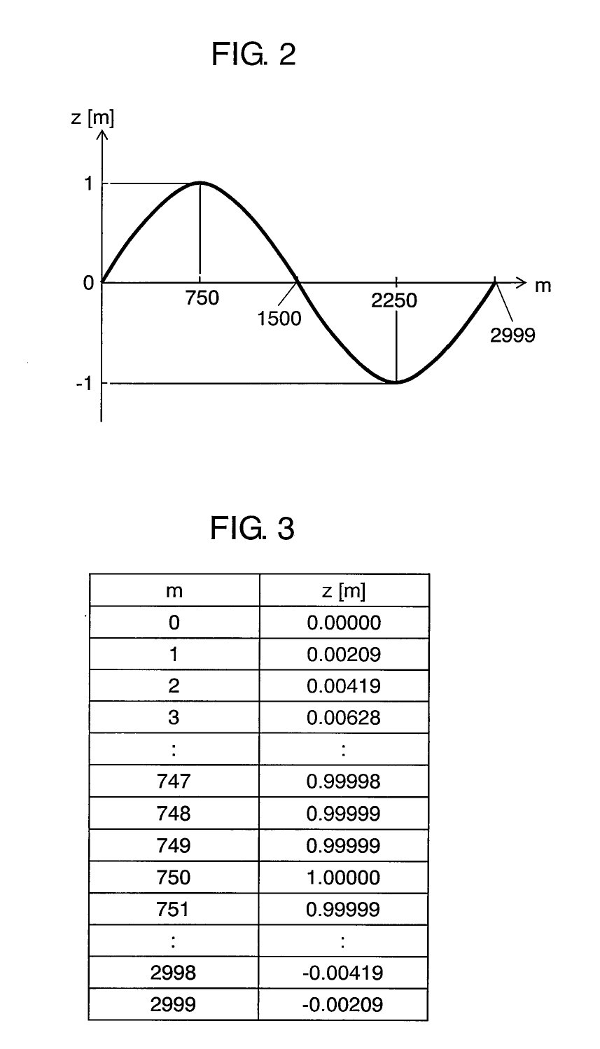

FIG. 2 is a graph showing an example of a sine wave table in the active noise controller in accordance with the first exemplary embodiment of the present invention, which is visually shown for easy understanding. FIG. 3 shows an example of the sine wave table in the active noise controller by using an example of numeric values as stored in a microprocessor for operation in accordance with the first exemplary embodiment of the present invention. FIGS. 2 and 3 show an example in which N is 3000.

Characteristic table 4 holds, in the memory, lower limit point PP1[f] and upper limit point PP2[f] for processing error signal ε[n] (f denotes a frequency (Hz)) based on the phase characteristic as a transfer characteristic from loudspeaker 10 to microphone 11.

When the phase characteristic at f(Hz) is θ[f] (°), the following relational expressions (6) are satisfied.

PP1[f]=N×θ[f]/360+α

PP2[f]=PP1[f]+β (6)

Herein, α and β may be any positive constants but they need to satisfy α<N/4 and β+α<N/4.

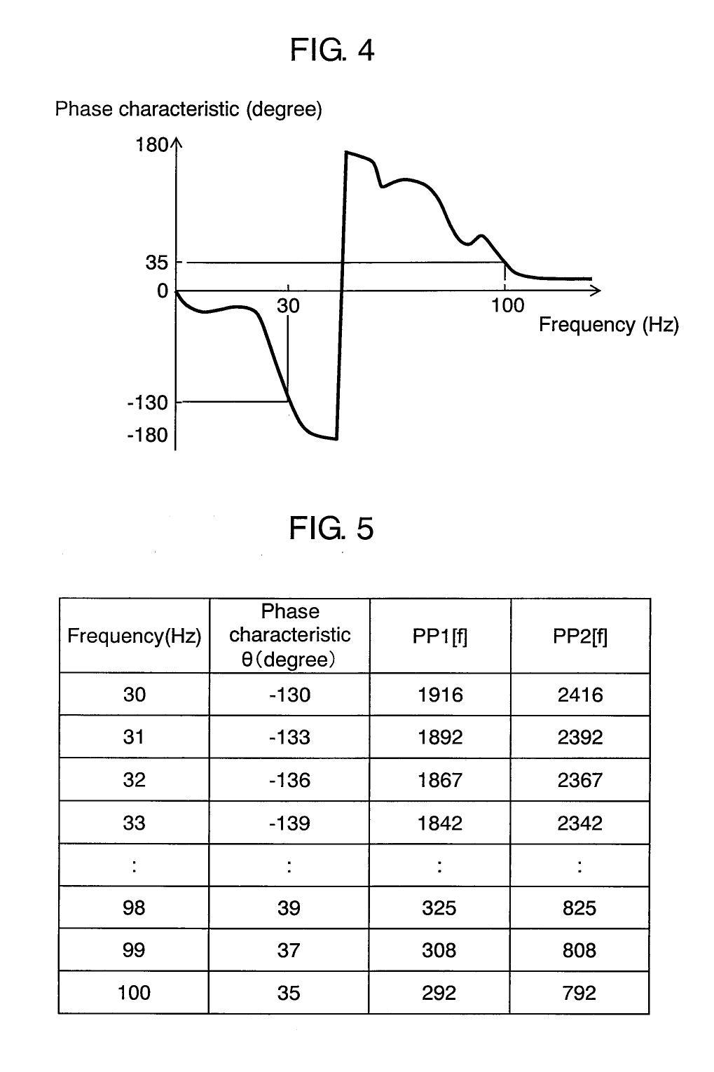

FIG. 4 is a graph showing an example of a transfer characteristic from a loudspeaker to a microphone of the active noise controller in accordance with the first exemplary embodiment of the present invention. FIG. 5 shows an example of a characteristic table having a lower limit passing point and an upper limit passing point corresponding to the transfer characteristic from the loudspeaker to the microphone shown in FIG. 4 in the active noise controller in accordance with the first exemplary embodiment of the present invention. FIG. 5 shows lower limit PP1[f] of the readout point and upper limit PP2 [f] of the readout point in which N is 3000 and frequency f of noise to be controlled ranges from 30 Hz to 100 Hz.

Sine wave generating unit 5 stores the current readout position P[n] of sine wave table 3 in the memory, and moves the current readout position at every cycle by formula (7) based on frequency f of noise to be controlled.

P[n+1]=P[n]+N×f×T (7)

When the calculation result in the right side of the formula (7) is not less than N, a value obtained by subtracting N from the calculation result in the right side of the formula (7) is defined as P[n+1].

At the same time, sine wave generating unit 5 generates basic sine wave signal x1[n] having the same frequency as frequency f of noise to be controlled by the following formulae (8) and (9).

i×1=P[n] (8)

x1[n]=z[i×1] (9)

When the calculation result in the right side of the formula (8) is not less than N, a value obtained by subtracting N from the calculation result in the right side of the formula (8) is defined as i×1.

Furthermore, cosine wave generating unit 6 generates basic cosine wave signal x2[n] having the same frequency as frequency f of noise to be controlled and preceding from basic sine wave signal x1[n] by ¼ by the following formulae (10) and (11).

i×2=P[n]+N/4 (10)

x2[n]=z[i×2] (11)

When the calculation result in the right side of the formula (10) is not less than N, a value obtained by subtracting N from the calculation result in the right side of the formula (10) is defined as i×2.

At the same time, coefficient update signal generating unit 14 reads lower limit point PP1[f] and upper limit point PP2[f] in frequency f of noise to be controlled from characteristic table 4 based on frequency f of noise to be controlled; based on them, processes error signal ε[n] detected by microphone 11 by formulae (3) and (4); and generates coefficient update signals ε1[n] and ε2[n], respectively.

First and second one-tap digital filters 7 and 8 generate first and second control signals y1[n] and y2[n] by formulae (12) and (13), respectively.

y1[n]=W1[n]×x1[n] (12)

y2[n]=W2[n]×x2[n] (13)

First and second adaptive control algorithm operating units 12 and 13 update filter coefficients W1[n] and W2[n] held by first and second one-tap digital filters 7 and 8 by formulae (14) and (15), respectively.

W1[n+1]=W1[n]−ε1[n] (14)

W2[n+1]=W2[n]−ε2[n] (15)

Filter coefficient W1[n] and filter coefficient W2[n] are allowed to converge by using the above-mentioned procedure. Thus, noise to be controlled can be reduced.

Herein, a mechanism for reducing noise having a frequency to be controlled by coefficient updating formulae (14) and (15) is described.

A noise controller described in a conventional example sequentially updates filter coefficients W1[n] and W2[n] based on an LMS (Least Mean Square) algorithm. The followings are the updating formulae.

W1[n+1]=W1[n]−μ×r1[n]×ε[n] (1)

W2[n+1]=W2[n]−μ×r2[n]×ε[n] (2)

Thus, in general, the product of error signal ε[n] and a sine wave signal and a cosine wave signal having a frequency of noise to be controlled as reference sine wave signal r1[n] and reference cosine wave signal r2[n] is used. This uses the orthogonal property between the sine wave and the cosine wave. In the sequential update for a long period of time (that is, n→∞), the product of frequency components having the same frequency as that of reference sine wave signal r1 and reference cosine wave signal r2 in error signal ε is accumulated, and the accumulated values of the product of the other frequency components becomes 0. Thus, W1[n] and W2[n] are updated so that frequency components having the same frequency as that of reference sine signal r1 and reference cosine signal r2 in error signal ε are reduced. Finally, when the frequency components having the same frequency as the frequency of the reference sine wave signal and the reference cosine wave signal in the error signal ε become 0, and average coefficient update of W1[n] and W2[n] becomes 0, and W1[n] and W2[n] converge.

On the other hand, the present invention does not use so-called reference signals (r1[n] and r2[n]). The present invention updates coefficients by using only coefficient update signals ε1[n] and ε2[n] generated from error signal ε[n] by formulae (3) and (4).

The ε1[n] and ε2[n] can be also represented as follows:

ε1[n]=1×ε[n] when PP1[f]≦P[n]≦PP2[f] is satisfied, and

ε1[n]=−1×ε[n] when PP1[f]+N/2≦P[n]≦PP2[f]+N/2 is satisfied.

In the cases other than the above, the following equation is satisfied.

ε1[n]=0×ε[n] (16)

Similarly,

ε2[n]=1×ε[n] when PP1[f]+N/4≦P[n]≦PP2[f]+N/4 is satisfied, and

ε2[n]=−1×ε[n] when PP1[f]+N×¾≦P[n]≦PP2[f]+N×¾ is satisfied.

In the cases other than the above, the following equation is satisfied.

ε1[n]=0×ε[n] (17)

In other words, ε1[n] and ε2[n] have frequency f of noise to be controlled that is the same as that of ε[n], which are equivalent to the product of square waves having one amplitude around 0. When the square wave signal at the side of ε1[n] is represented by H1[n] and the square wave signal at the side of ε2[n] is represented by H2[n], they can be represented as follows.

ε1[n]=H1[n]×ε[n] (18)

ε2[n]=H2[n]×ε[n] (19)

Herein, formulae (16) and (17) show that H1[n] and H2[n] are different from each other by ¼ cycle.

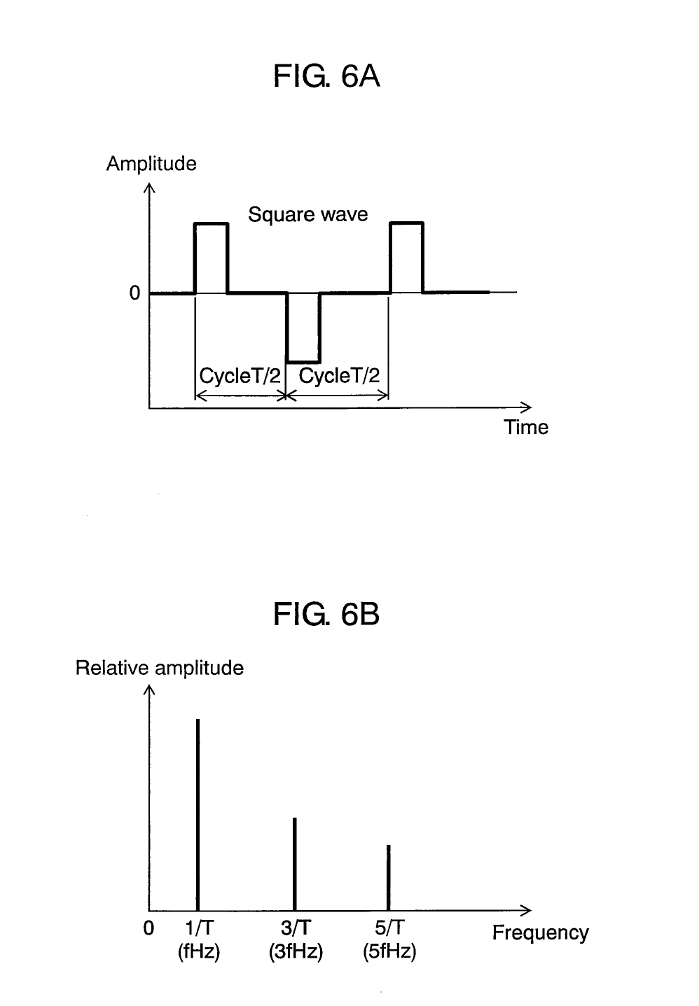

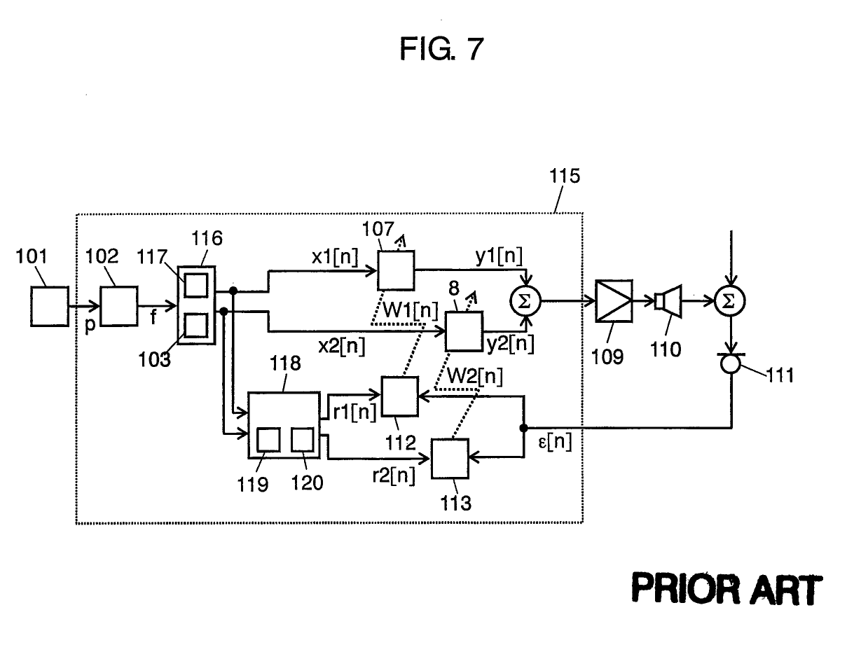

FIG. 6A is a graph showing a time-base waveform of square wave signal H1[n] (H2[n]) formed for processing an error signal in the active noise controller in accordance with the first exemplary embodiment of the present invention. FIG. 6B is a graph showing a harmonic analysis of square wave signal H1[n] (H2[n]) formed for processing an error signal in the active noise controller in accordance with the first exemplary embodiment of the present invention. FIGS. 6A and 6B show that each of square wave signals H1[n] and H2[n] includes a fundamental frequency component and odd-order higher harmonic wave. These are generally represented by the following formulae.

H1[n]=A1 Sin(2πfn/T)+A2 Sin(2π×3fn/T)+A3 Sin(2π×5fn/T) (20)

H2[n]=A1 Cos(2πfn/T)+A2 Cos(2π×3fn/T)+A3 Cos(2π×5fn/T) (21)

On the other hand, when coefficient updating formulae (16) and (17) of the digital filter are modified and the relations represented by the formulae (20) and (21) are substituted, the following equations are obtained:

ΔW1=W1[n+1]−W1[n]=−ε[n]×H1[n]

ΔW2=W2[n+1]−W2[n]=−ε[n]×H2[n]

W1=ΣΔW1=Σ(−ε[n]×H1[n]) (22)

W2=ΣΔW2=Σ(−ε[n]×H2[n]) (23)

wherein W1 and W2 are proportional to the accumulated values of (−ε[n]×H1[n]) and (−ε[n]×H2[n]), respectively.

Herein, when ε[n] is sine wave Sin(2πfn/T) with frequency f, W1 is represented as follows from formulae (20) and (22).

$$\begin{matrix}W 1 = Σ ( - ɛ [ n ] \times H 1 [ n 1 ] ) \\ = Σ \{ - Sin ( 2 π fn / T ) \times ( \begin{matrix}A 1 Sin ( 2 π fn / T ) + \\ A 2 Sin ( 2 π \times 3 fn / T ) + \\ A 3 Sin ( 2 π \times 5 fn / T ) + …\end{matrix} ) \}\end{matrix}$$

Furthermore, since the accumulated value of components having different frequencies becomes 0 due to the orthogonal property of sine waves, the following formula is satisfied.

$$\begin{matrix}W 1 = Σ ( - ɛ [ n ] \times H 1 [ n ] ) \\ = Σ ( - Sin ( 2 π fn / T ) \times A 1 Sin ( 2 π fn / T ) [ n ] )\end{matrix} \qquad ( 24 )$$

The same is true to W2. That is to say, in both W1 and W2, the product of only components with frequency f is accumulated, which is equal to the conventional ones using sine waves for reference signal. Coefficients W1 and W2 converge so that the noise with frequency f is reduced. Thus, similar to the conventional example using the sine wave for reference signal, the present invention can also reduce noise having the intended frequency f.

Furthermore, the size of the difference between the upper limit point and the lower limit point (PP2[f]−PP1[f]) in the present invention can be selected arbitrarily. In other words, actually, they can be treated the same as μ (convergence coefficient) in a conventional example. That is to say, the larger PP2[f]−PP1[f] is, the higher the convergence rate is. Meanwhile, when PP2[f]−PP1[f] is small, the convergence rate becomes slow. Thus, the convergence rate can be adjusted by the size of the PP2[f]−PP1[f].

Herein, the method of the present invention and the method described in patent document 1 are compared with each other in terms of an operation load. The method described in patent document 1 uses basic sine wave signal correction value table 19 (a basic sine wave signal correction value in a frequency of f (Hz) is referred to as C1[f]) and reference cosine wave signal correction value table 20 (a basic cosine wave signal correction value in a frequency of f (Hz) is referred to as C2[f]) simulating a transfer characteristic value from loudspeaker 10 to microphone 11 so as to generate reference sine wave signal r1[n] and reference cosine wave signal r2[n] by formulae (25) and (26), respectively.

r1[n]=C1[f]×x1[n]+C2[f]×x2[n] (25)

r2[n]=C1[f]×x2[n]−C2[f]×x1[n] (26)

Firstly, formulae (25) and (26) include two multiplications respectively while the present invention does not need multiplication because it does not use a reference signal. Furthermore, also in the update of coefficients, the method described in patent document 1 needs two multiplications as shown in formulae (1) and (2). Meanwhile, the present invention does not need multiplication as is apparent by formulae (14) and (15).

Thus, the method described in patent document 1 needs four multiplications in every sampling cycle in order to obtain coefficients W1 and W2, respectively. However, the present invention does not need multiplication. Therefore, according to the active noise controller in accordance with the present invention, an operation load can be reduced as compared with the method described in patent document 1.

Furthermore, in the present invention, by preparing a plurality of first and second one-tap digital filters 7 and 8 and a plurality of first and second adaptive control algorithm operating units 12 and 13, respectively, multiple-order components of noise to be controlled can be suppressed.

### INDUSTRIAL APPLICABILITY

An active noise controller in accordance with the present invention can reduce an operation load by minimizing the execution of product-sum operation, and thus it is useful as a practical and low-cost active noise controller.

1. An active noise controller comprising:
a frequency detecting unit for detecting a frequency of noise to be controlled from a noise source;
a sine wave generating unit for generating a sine wave having a same frequency as the frequency of the noise detected by the frequency detecting unit;
a cosine wave generating unit for generating a cosine wave having a same frequency as the frequency of the noise detected by the frequency detecting unit;
a first one-tap digital filter to which a sine wave signal from the sine wave generating unit is input;
a second one-tap digital filter to which a cosine wave signal from the cosine wave generating unit is input;
an interference signal generating unit to which a noise control signal obtained by adding an output from the first one-tap digital filter and an output from the second one-tap digital filter to each other is input and which outputs an interference signal to cause interference with the noise from the noise source;
an error signal detecting unit for detecting an error signal generated as a result of the interference of the interference signal output from the interference signal generating unit and the noise from the noise source;
a first coefficient updating unit for updating a filter coefficient of the first one-tap digital filter; and
a second coefficient updating unit for updating a filter coefficient of the second one-tap digital filter,
wherein the first coefficient updating unit and the second coefficient updating unit update coefficients of the first one-tap digital filter and the second one-tap digital filter so that noise in the error signal detecting unit is reduced, based on a coefficient update signal obtained by processing the error signal from the error signal detecting unit with ½ cycle of the frequency of the noise for a predetermined period.
